(19) United States
(12) Patent Application Publication
(10) Pub. No.: US 2021/0378926 A1
(43) Pub. Date: Dec. 9, 2021

(54) A TOPICAL COMPOSITION

(71) Applicant: CONOPCO, INC., D/B/A UNILEVER, TRUMBULL, CT (US)

(72) Inventor: MAYA TREESA SAJI, Bangalore (IN)

(73) Assignee: CONOPCO, INC., D/B/A UNILEVER, TRUMBULL, CT (US)

(21) Appl. No.: 17/282,813

(22) PCT Filed: Oct. 17, 2019
(86) PCT No.: PCT/EP2019/078179

§ 371 Date: Apr. 5, 2021

(30) Foreign Application Priority Data
Oct. 24, 2018 (EP) 18202218.6

(51) Int. Cl. A61K 8/34; A01N 31/08; A01N 59/16; A01N 31/04; A61K 8/19; A61Q 17/00; A01P 1/00

(52) U.S. Cl. CPC A61K 8/347; A01N 31/08; A01N 59/16; A01P 1/00; A61K 8/19; A61Q 17/005; A01N 31/04

(57) ABSTRACT

The present invention relates to a topical composition. More particularly the present invention relates to antimicrobial composition for providing benefits against malodor. Accordingly, the present invention provides a topical composition comprising; a. at least one compound selected from a biphenol; and, b. at least one coinage metal compound.

### FIELD OF THE INVENTION

The present invention relates to a topical composition. More particularly the present invention relates to an antimicrobial composition for benefits against malodor.

### BACKGROUND OF THE INVENTION

People try to take good care of the external surfaces of their body as well as those of their pets to enable overall good health. Specific skin related issues that people care about include, good skin health free of infections, good skin tone, free from malodour and adequate moisturization.

A good health for external surfaces including skin, oral cavity and scalp care are typically achieved by keeping them free of trouble causing microorganisms. This is preferably done by the application of antimicrobial compounds that can kill the harm causing microorganism or inhibit their growth. Some of the harmful microorganisms that commonly found on human skin are Escherichia coli (E. coli), Staphylococcus aureus (S. aureus), Staphylococcus hominis (S. hominis) etc. Bacteria like E. coli and S. aureus per se do not trigger a pathogenic effect whilst commonly present on the skin. However, when they enter the human body through cuts on the skin and through acts like ingestion, these bacteria become pathogenic. On the hand presence of bacteria like S. hominis in presence of skin biproduct e.g. sebum produces malodor.

Malodour is a common problem as all types of skin invariably produces biproduct like sebum which in presence of microorganisms as described above causes malodour. Therefore, for researcher this has been an interesting field for exploration of different actives either alone or in combinations for providing effective protection against malodour formation on the skin.

There are few prior arts which discloses composition to fight against malodour.

U.S. Pat. No. 6,080,391 discloses the use of one or more oxidoreductases in combination with a mediator for the reduction of malodour. Malodour reducing compositions and products comprising such composition are also disclosed.

EP1093354 discloses Cosmetic method for reducing or preventing body malodour by topically applying to human skin an active agent capable of inactivating body malodour-causing microorganisms comprising corynebacteria, characterised in that the agent is capable of inactivating, of the corynebacteria, only those corynebacteria capable of catabolising fatty acids.

WO2009101615 discloses A method of controlling body odour. The method comprises topically administering to a subject an effective amount of at least one species of a lytic bacteriophage capable of killing odor-generating bacteria. Deodorant compositions comprising such bacteriophages are also disclosed.

The available arts mainly focused on using some other good microbes to provide protection against malodour. A simple and easy method for handling malodour is yet to be found.

Furthermore, people are always on a look out for new technologies e.g. actives or combination of actives that delivers improved protection against malodour. Further, it is preferred if minimal amounts of known antimicrobial actives are used as people nowadays prefer minimum exposure to chemical ingredients. Therefore, antimicrobial compositions and actives that deliver benefits against malodour, remains a topic of interest.

Need therefore exists to provide an antimicrobial composition comprising one or more actives that delivers a simple and easy way of handling malodour.

It is therefore an object of the present invention to provide a topical composition.

It is another object of the present invention to provide an antimicrobial composition for providing effective protection against malodor.

It is yet another object of the present invention to provide a topical composition for benefit against malodour.

The present inventors have surprisingly found out that a combination comprises a biphenol and a coinage metal compound provides good benefits against malodor and thereby satisfying one or more of the above-mentioned objects.

### SUMMARY OF THE INVENTION

Accordingly, in a first aspect there is provided a topical composition comprising;
a. at least one compound selected from a biphenol; and,
b. at least one coinage metal compound.

In a second aspect there is provided a method of disinfecting a surface comprising the step of applying on to the surface a composition of the first aspect.

In a third aspect there is provided use of a topical composition comprising (a) at least one compound selected from a biphenol; and, (b) at least one coinage metal compound for obtaining benefit against malodour.

These and other aspects, features and advantages will become apparent to those of ordinary skill in the art from a reading of the following detailed description. For the avoidance of doubt, any feature of one aspect of the present invention may be utilized in any other aspect of the invention. The word “comprising” is intended to mean “including” but not necessarily “consisting of” or “composed of.” In other words, the listed steps or options need not be exhaustive. It is noted that the examples given in the description below are intended to clarify the invention and are not intended to limit the invention to those examples per se. Similarly, all percentages are weight/weight percentages unless otherwise indicated. Except in the operating and comparative examples, or where otherwise explicitly indicated, all numbers in this description indicating amounts of material or conditions of reaction, physical properties of materials and/or use are to be understood as modified by the word “about”. Numerical ranges expressed in the format “from x to y” are understood to include x and y. When for a specific feature multiple preferred ranges are described in the format “from x to y”, it is understood that all ranges combining the different endpoints are also contemplated.

### DETAILED DESCRIPTION OF THE INVENTION

The composition of the present invention intended for topical application i.e. application predominantly on human skin. The most preferred application of the composition is as an antimicrobial composition. Antimicrobial composition as mentioned herein above preferably means any composition, which is capable of killing or at least cause substantial reduction of the common disease causing and or foul smell causing microbes. The common disease causing gram-positive organisms includes Staphylococcus, Streptococcus and Enterococcus spp. Some of common disease causing gram-negative organisms includes Escherichia coli, Salmonella, Klebsiella and Shigella. Escherichia coli and Salmonella can cause severe gastrointestinal illnesses. One of the foul smell causing bacterial exist on human skin is S. hominis.

The present invention relates to a topical composition comprising;
a. at least one compound selected from a biphenol; and,
b. at least one coinage metal compound.

### Biphenol

The composition as per the present invention (the composition) comprises at least one biphenol.

Examples of biphenol that are suitable for use in the present invention include 2,4′-biphenol (IUPAC name: 2,4′-Dihydroxybiphenyl), 2,2′-biphenol (2,2′-Dihydroxybiphenyl), 3,3′-biphenol (3,3′-Dihydroxybiphenyl), 4,4′-biphenol (4,4′-Dihydroxybiphenyl), and mixtures thereof.

Preferably, the biphenol is a diallylbiphenol. This means the biphenol comprises diallyl substitution. The diallyl substitution may be present at any of the positions on biphenol structure. The preferred diallylbiphenol is a (5-3′-diallyl) biphenol or a (5-5′-diallyl) biphenol. The most preferred diallylbiphenol is a (5-3′-diallyl) biphenol.

The most preferred biphenol are selected from honokiol and/or magnolol.

A combination of two or more biphenol are also within the scope of the present invention. When two or more biphenol are used, the most preferred combination is honokiol and magnolol.

Honokiol and magnolol both present or isolated from bark, seed cones and leaves belonging to the genus magnolia. Bark being the most prominent source of honokiol and magnolol.

When the invention employs a combination of honokiol and magnolol, the bar extract from the genus magnolia may also preferably be used. The amount of magnolia bark extract in that case to be calculated based on its honokiol and magnolol content.

Honokiol has the following structure:

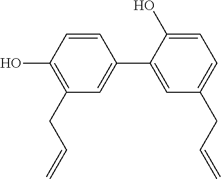 [SMILES: C=CCc1ccc(O)c(-c2ccc(O)c(CC=C)c2)c1]

Magnolol has the following structure:

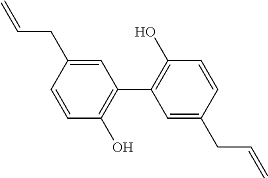 [SMILES: C=CCc1ccc(O)c(-c2cc(CC=C)ccc2O)c1]

Preferably, the composition comprises from 0.001 to 10%, preferably from 0.005 to 8%, more preferably from 0.01 to 6%, even more preferably from 0.05 to 5%, furthermore preferably from 0.1 wt to 4%, still more preferably from 0.5 to 3% and yet more preferably from 1 to 1.5% by weight of a biphenol.

### Coinage Metal Compound

Coinage metal preferably refers to those metal which are placed in group 11 of the periodic table. The coinage metal compound as referred in the present invention preferably refer to the compounds of coinage metal of group 11 of the periodic table. The preferred coinage metal compound is selected from compound of copper, silver or gold. The most preferred ones are copper and silver because the ease of availability and cost. The further most preferred coinage metal is silver because of its inherent antimicrobial property. A combination of two or more coinage metal compounds are also within the purview of the present invention.

The preferred silver salt is selected from the group consisting of silver oxide, silver nitrate, silver acetate, silver sulfate, silver benzoate, silver salicylate, silver carbonate, silver citrate, silver phosphate or mixtures thereof.

Alternatively, the silver compound can also be a complex of silver. The silver compound also may preferably be a complex of silver. The silver complex may be formed by reacting silver with one or more of a chelating agent. Chelates are characterized by coordinate covalent bonds. These occur when unbonded pairs of electrons on non-metal atoms like nitrogen and oxygen fill vacant d-orbitals in the metal atom being chelated. Valence positive charges on the metal atom can be balanced by the negative charges of combining amino acid ligands. The bonding of an electron pair into vacant orbitals of the metal allows for more covalent bonding than the valence (or oxidation number) of the metal would indicate. Forming bonds this way is called coordination chemistry. This allows chelates to form, providing that the ligands can bond with two or more moieties within the same molecule and providing that proper chemistry promoting chelation is present. An important factor is the strength of the complex formed between the metal ion and the chelating agent. This determines whether the complex will be formed in the presence of competing anions. The stability or equilibrium constant (K), expressed as log K, has been determined for many metals and chelating agents. The higher the log K values, the more tightly the metal ion will be bound to the chelating agent and the more likely that the complex will be formed.

Preferred chelating agents are ethylene diamine tetraacetic acid (EDT A), ethylene diamine dissuccinate (EDDS), N, N-bis (carboxymethyl) glutamic acid (GLDA), Diethylenetriaminepentaacetic acid (DTPA), Nitrilotriacetic acid (NTA) and Ethanoldiglycinic acid ((EDG). DTPA is particularly preferred and especially in combination with Silver. Chelating agents are usually used in the form of their salts with a metal. For example, EDTA is used in the form of disodium or tetrasodium salt.

Accordingly, it is preferred to use a salt form of a chelating agent over the natural acid form. Preferably, the molar ratio of silver to the chelating agent is 1:0.25 to 1:10, more preferably 1:0.5 to 1:5 and most preferably 1:1 to 1:3.

The amount of silver as mentioned is irrespective of its oxidation state.

Preferably, in the disclosed antimicrobial cleansing composition silver compound is present at levels not less than 0.4 ppm, still preferably not less than 0.5 ppm and further preferably not less than 1 ppm and it is preferred that the silver compound in the composition is present at levels not more than 80 ppm, more preferably not more than 50 ppm, further preferably not more than 20 ppm and still further preferably not more than 10 ppm and most preferably not more than 5 ppm. It is highly preferred that the silver compound in the antimicrobial cleansing composition is present at 0.5 to 5 ppm.

The composition of the present invention further preferably comprises at least one essential oils selected from thymol, terpineol, eugenol, geraniol, carvacrol or their analogue.

Essential oil preferably used at a concentration in the range of 0.001 to 10%, more preferably 0.01 to 5%, furthermore preferably 0.1 to 3% by weight of the composition.

Preferably the essential oil is selected from a combination of thymol, terpineol and eugenol and most preferably the combination is thymol and terpineol.

The preferred use of the composition is in the form of a cream, lotion, spray or deodorant/antiperspirant. The most preferred use of the composition is in the form of deodorant/antiperspirant.

When the composition of the present invention in the form of an antiperspirant compositions may advantageously comprise additional ingredients. Such ingredients include any other antiperspirant active, skin care agents such emollients, humectants and skin barrier promoters; skin appearance modifiers such as skin lightening agents and skin smoothing agents; anti-microbial agents, in particular organic anti-microbial agents, and preservatives.

The anti-perspirant active can be applied cosmetically and topically to the skin, broadly speaking, by one of two methods. Different consumers prefer one method or the other. In one method, sometimes called a contact method, a composition is wiped across the surface of the skin, depositing a fraction of the composition as it passes. In the second method, sometimes called the non-contact method, the composition is sprayed from a dispenser held proximate to the skin, often in an area of about 10 to 20 cm². The spray can be developed by mechanical means of generating pressure on the contents of the dispenser, such as a pump or a squeezable sidewall or by internally generated pressure arising from a fraction of a liquefied propellant volatilizing, the dispenser commonly being called an aerosol.

There are broadly speaking two classes of contact compositions, one of which is liquid and usually applied using a roll-on dispenser or possibly absorbed into or onto a wipe, and in the second of which the antiperspirant active is distributed within a carrier liquid that forms a continuous phase that has been gelled. In one variation, the carrier fluid comprises a solvent for the antiperspirant and in a second variation, the antiperspirant remains a particulate solid that is suspended in an oil, usually a blend of oils.

### Stick or Soft Solid Compositions

Many different materials have been proposed as gellant for a continuous oil phase, including waxes, small molecule gelling agents and polymers. They each have their advantages and of them, one of the most popular class of gellant has comprised waxes, partly at least due to their ready availability and ease of processing, including in particular linear fatty alcohol wax gellants. A gelled antiperspirant composition is applied topically to skin by wiping it across and in contact with the skin, thereby depositing on the skin a thin film.

The nature of the film depends to a significant extent on the gellant that is employed. Although wax fatty alcohols have been employed as gellant for many years, and are effective for the purpose of gelling, the resultant product is rather ineffective at improving the visual appearance of skin, and in particular underarm skin, to which the composition has been applied. This problem has been solved by including ameliorating materials for example, di or polyhydric humectants and/or a triglyceride oil.

### Roll-On

Liquid compositions that are applicable from a roll-on broadly speaking can be divided into two classes, namely those in which an antiperspirant active is suspended in a hydrophobic carrier, such as a volatile silicone and those in which the antiperspirant active is dissolved in a carrier liquid. The latter has proven to be more popular. There are mainly two sorts of dissolving carrier liquid, namely carriers that are predominantly alcoholic, which is to say the greater part of the dissolving carrier fluid comprises ethanol and the second class in which the carrier liquid is mainly water. The former was very popular because ethanol is a mild bactericide in its own right, but its popularity waned because it stings, especially if the surface onto which the composition has been applied has been damaged or cut, such as can easily arise during shaving or other de-hairing operations.

The second class of formulations that is an alternative to alcoholic formulations comprise a dispersion of water-insoluble or very poorly water soluble ingredients in an aqueous solution of the antiperspirant. Herein, such compositions will be called emulsions. Antiperspirant roll-on emulsions commonly comprise one or more emulsifiers to maintain a distribution of the water-soluble ingredients.

### Aerosol Compositions

The antiperspirant composition may be delivered through an aerosol composition which may comprise a propellant in addition to the other ingredients described hereinabove. Commonly, the propellant is employed in a weight ratio to the base formulation of from 95:5 to 5:95. Depending on the propellant, in such aerosol compositions the ratio of propellant to base formulation is normally at least 20:80, generally at least 30:70, particularly at least 40:60, and in many formulations, the weight ratio is from 90:10 to 50:50. A ratio range of from 70:30 to 90:10 is sometimes preferred.

Propellants herein generally are one of three classes; i) low boiling point gasses liquified by compression, ii) volatile ethers and iii) compressed non-oxidising gases.

Class i) is conveniently a low boiling point material, typically boiling below −5° C., and often below −15° C., and in particular, alkanes and/or halogenated hydrocarbons. This class of propellant is usually liquefied at the pressure in the aerosol canister and evaporates to generate the pressure to expel the composition out of the canister. Examples of suitable alkanes include particularly propane, butane or isobutane. The second class of propellant comprises a very volatile ether of which the most widely employed ether hitherto is dimethyl ether. This propellant can advantageously be employed at relatively low weight ratio of propellant to base formulation, for example to as low as 5:95. It can also be employed in admixture with, for example, compressible/liquefiable alkane gasses. The third class of propellant comprises compressed non-oxidising gasses, and in particular carbon dioxide or nitrogen. Inert gases like neon are a theoretical alternative.

When the composition of the invention is delivered in a roll-on, a firm solid or a stick format, the topically acceptable carrier comprises a hydrophobic carrier or an aqueous carrier. The hydrophobic carrier in such cases may comprise a silicone compound, low boiling alcohol or a wax. When the composition comprises a propellant it is delivered as an aerosol.

The composition of the present invention can comprise a wide range of other optional components. The CTFA Personal care Ingredient Handbook, Second Edition, 1992, which is incorporated by reference herein in its entirety, describes a wide variety of non-limiting personal care and pharmaceutical ingredients commonly used in the skin care industry, which are suitable for use in the compositions of the present invention. Examples include: antioxidants, binders, biological additives, buffering agents, colorants, thickeners, polymers, astringents, fragrance, conditioners, exfoliating agents, pH adjusters, preservatives, natural extracts, essential oils, skin sensates, skin soothing agents, and skin healing agents.

The present invention also provides a topical composition for antimicrobial benefits.

Further provided a method of disinfecting a surface comprising the steps of applying on to the surface a composition of the invention.

The present invention also provides use of a topical composition comprising (a) at least one compound selected from a biphenol; and, (b) a coinage metal compound for obtaining benefit against malodour.

The present invention also provides use of a combination of at least one biphenol and at least one coinage metal compound as a malodour reducing agent in a topical composition.

The above use can preferably be therapeutic or non-therapeutic. Most preferably the use of the composition of the present invention for non-therapeutic benefits.

Now the invention will be demonstrated in terms of examples. The following examples are just for illustration and in no way limits the scope of the present invention.

### EXAMPLES

The efficacy of different ingredients and combinations were tried using the Malodor Assay protocol with S. hominis (ATCC 27844) as described below:

The malodour assay was done in a 96 well plate (Nunc™, ThermoFisher). First, a lead acetate paper was prepared as follows. A Whatman™ (No. 1) filter paper (150 mm, Cat No. 1001-150) was taken and cut to match the top cross-sectional dimension of the 96 well plate. 1% Lead acetate (Hi Media, RM 757) solution was prepared in distilled water and the Whatman™ filter paper was soaked in the prepared lead acetate solution. The excess solution from the filter paper was drained and allowed to dry in a Laminar Air Flow for 1 hour. After drying, the filter paper was wrapped in an Aluminium foil and autoclaved at 121° C. for about 20 minutes. This lead acetate paper was used for the assay to capture the malodour generation at the later stage.

A TSB broth solution was prepared by adding 0.1% L-cysteine-HCl (Sigma, C1276) into TSB (Bacto, 211825, 30 g/L) and autoclaved. After that, the 96 well plate was taken followed by addition of 180 μL/well of the above prepared TSB solution into the wells. Then 20 μL/well of S. hominis culture (10-8 CFU/mL) was added to the wells. Different ingredient and/or mix of ingredients as per following Table 1 were added into the wells along with a control (only distilled water and bacteria) as per the required concentration (as mentioned in Table 1). Finally, the lead acetate paper that was prepared previously was placed on the wells of the 96-well plate and the lid was closed. This plate was then incubated in an incubator (LABTOP, bacteriological incubator) at 37° C. for about 16 hours. The lid of the plate was opened after the incubation and observed the lead acetate paper for any visual change in colour. Visible blackening of lead acetate shows lead sulfide formation and indicate the presence of H₂S. The results and the effect of actives were compared with the control (completely black).

The test samples were used as per the Table 1:

TABLE 1

| Example | Ingredients and amounts (in ppm) | | | | | | |
|---|---|---|---|---|---|---|---|
| No. | HM | H | M | Silver* | FeSO4 | CuSO4 | TT |
| A | 5 | | | | | | |
| B | 10 | | | | | | |
| C | | 5 | | | | | |
| D | | | 10 | | | | |
| E | | | | 1 | | | |
| F | | | | 2 | | | |
| G | | | | | 100 | | |
| H | | | | | 200 | | |
| I | | | | | 500 | | |
| J | | | | | | 1 | |
| K | | | | | | 2 | |
| L | | | | | | | 0.01 |
| M | 5 | | | | 100 | | |
| N | 10 | | | | 200 | | |
| 1 | 5 | | | 1 | | | |
| 2 | 10 | | | 2 | | | |
| 3 | 5 | | | | | 1 | |
| 4 | 10 | | | | | 2 | |
| 5 | | 5 | | 2 | | | |
| 6 | | | 10 | 2 | | | |
| 7 | | 1 | | 0.5 | | | 0.01 |
| 8 | | | 2 | 0.5 | | | 0.01 |

*the silver used here as Silver DTPA complex.

In the above table HM means a combination of honokiol and magnolol at 1:1 ratio which was purchased from World-Way Biotech Inc.®, China. In the same way ‘H’ means only honokiol and ‘M’ means only magnolol. In this case Honokiol (98% pure) and Magnolol (98% pure) was purchased from World-Way Biotech Inc.®, China. In the above table ‘TT’ means a combination of thymol and terpineol at 1:1 ratio. Thymol and terpineol was purchased from Nishant Aroma®, India. FeSO4 was purchased from Merk® Cat. No: ML7M573164. CuSO4 was purchased from Merk® Cat. No: 17515.

The silver DTPA complex was prepared by using the following protocol:

The silver DTPA complex as mentioned above was prepared by using 1.500 g of Silver oxide powder with 22.5 g of 40% Na₅DTPA (Sodium salt of diethylene triamine pentaacetic acid). The above mixture was stirred and heated at ˜45° C. in a water bath for 10 minutes. Any particulates observed are broken with glass rod. After that 975 g of water was added water stirring ambient temp (˜25° C.). The stirring was continued for 10 minutes. After that 0.8 g of powdered lauric acid was added and stirred for 30 minutes. The resulting mixture was centrifuged to separate out the supernatant from the residue for 5 minutes. The supernatant is silver DTPA complex used in the experiments.

The results of these experiments are expressed in terms of “% reduction” in H₂S production which is determined from the blackening of the spot as explained above. Each spot with different test samples has been compared with the control (completely black) sample. It was then rated on a scale of 0 to 10 (10 being completely black as like control and 0 is no blackening). From the score the percentage reduction was evaluated e.g. if the blackening of any sample is as dark as control then the score given was “8-10” and accordingly % reduction is <20%.

The results of the experiments are summarized below:

TABLE 2

| Example Number | % reduction in H₂S production |
|---|---|
| A | 20-40% |
| B | 20-40% |
| C | 20-40% |
| D | <20% |
| E | <20% |
| F | 20-40% |
| G | <20% |
| H | <20% |
| I | <20% |
| J | <20% |
| K | 20-40% |
| L | <20% |
| M | 20-40% |
| N | 20-40% |
| 1 | 80-100% |
| 2 | 80-100% |
| 3 | 80-100% |
| 4 | 80-100% |
| 5 | 80-100% |
| 6 | 60-80% |
| 7 | 80-100% |
| 8 | 60-80% |

From the above table 2 it is evident that “% reduction in H₂S production” for the examples (Example 1 to 8) that are within the scope of the present invention are much higher when compare with the examples (Example A to N) that are outside the scope of the present invention. Therefore, it is proved that the composition of the present invention reduces malodour quite efficiently.

1. A topical composition comprising;
a. at least one compound selected from a biphenol; and,
b. at least one coinage metal compound
wherein the biphenol is selected from 2,4′-biphenol or 2,2′ biphenol; and
wherein the coinage metal compound is a silver compound.

2. (canceled)

3. The topical composition as claimed in claim 1, wherein the biphenol comprises di-allyl substitution.

4. The topical composition as claimed in claim 2, wherein the di-allyl substitution is at the position 3-5′ or 5-5′ of the biphenol.

5. The topical composition as claimed in claim 3, wherein the biphenol is selected from honokiol and magnolol.

6. (canceled)

7. (canceled)

8. The topical composition as claimed in claim 1, wherein the silver compound selected from group consisting of silver oxide, silver nitrate, silver acetate, silver sulfate, silver benzoate, silver salicylate, silver carbonate, silver citrate, silver phosphate or combinations thereof.

9. The topical composition as claimed in claim 5, wherein the silver compound is a complex of silver.

10. The topical composition as claimed in claim 1, comprising at least one essential oil selected from thymol, terpineol, eugenol, geraniol, carvacrol or their analogues.

11. The topical composition as claimed in claim 7, wherein the essential oil comprises a combination of thymol and terpineol.

12. The topical composition as claimed in claim 1, wherein the topical composition is configured for antimicrobial benefits.

13. A method of disinfecting a surface comprising the step of applying on to the surface a topical composition as claimed in claim 1.

14. A method for obtaining a benefit against malodour, comprising applying the composition as claimed in claim 1 onto the surface.

15. (canceled)
